(19) United States
(12) Patent Application Publication
(10) Pub. No.: US 2015/0282782 A1
(43) Pub. Date: Oct. 8, 2015

(54) SYSTEM AND METHOD FOR DETECTION OF LESIONS

(71) Applicant: General Electric Company, Schenectady, NY (US)

(72) Inventors: Fei Zhao, Schenectady, NY (US); Vivek Prabhakar Vaidya, Bangalore (IN); Rakesh Mullick, Bangalore (IN); Soma Biswas, Bangalore (IN); Chuyang Ye, Baltimore, MD (US)

(73) Assignee: General Electric Company, Schenectady, NY (US)

(21) Appl. No.: 14/247,265

(22) Filed: Apr. 8, 2014

(51) Int. Cl. A61B 8/08

(52) U.S. Cl. CPC A61B 8/0825; A61B 8/5223; A61B 8/5207

(57) ABSTRACT

A method for detecting a lesion in an anatomical region of interest is presented. The method includes identifying one or more candidate mass regions in each of a plurality of 3D ultrasound images acquired at different view angles from the anatomical region of interest. Single-view features corresponding to each candidate mass region are identified. For a candidate mass region, a similarity metric between the single-view features corresponding to the candidate mass region and the single-view features corresponding to the other candidate mass regions is determined. The candidate mass region is classified based at least on the similarity metric. A system for imaging and a non-transitory computer readable media for detection of the lesion are also presented.

### BACKGROUND

Embodiments of the present specification relate to imaging, and more particularly to the identifying lesions in an anatomical region of interest using three-dimensional ultrasound imaging.

Cancer is one of the leading causes of death and breast cancer is a leading cause of death in women. Ultrasound imaging is used as an adjunct to mammography, serving as a screening tool to detect lesions such as breast masses and has gradually gained popularity. When compared with mammography, ultrasound imaging is less expensive and more sensitive to detecting abnormalities in dense breasts. In addition, ultrasound imaging introduces no radiation.

Typically, during the process of ultrasound imaging, a clinician attempts to capture one or more views of a certain anatomy to confirm or negate a particular medical condition. Once the clinician is satisfied with the quality of the view or the scan plane, the image is frozen for further manual analysis by the clinician. The clinician may then examine the image to manually detect the presence of lesion(s). However, the manual detection of lesions in the ultrasound images can be time consuming. To that end, Computer Aided Detection (CAD) solutions have been developed to aid in the automated detection of masses in breast tissues.

Currently, various CAD based solutions are available for analyzing two-dimensional (2D) ultrasound images. In such CAD based solutions, each of the 2D ultrasound images is analyzed individually in order to detect the lesions. However, these 2D ultrasound images provide a limited view of any anatomical region of interest.

Further, in recent years, CAD solutions have been used in connection with three-dimensional (3D) ultrasound imaging systems. Use of the 3D ultrasound imaging has reduced operator dependency in comparison to the 2D ultrasound imaging. To scan the entire breast using the 3D ultrasound imaging it is beneficial to acquire two to five images at different orientations. The 3D ultrasound images, thus captured, yield multiple views of the same tissue masses with overlapping regions. These 3D ultrasound images are then individually analyzed by the 3D ultrasound imaging system to determine the presence of any lesions. Such individual analysis of the 2D and/or 3D ultrasound images may lead to an increased number of false positive detections.

### BRIEF DESCRIPTION

In accordance with an embodiment of the present specification, a method for detecting a lesion in an anatomical region of interest is presented. The method includes receiving a plurality of three-dimensional ultrasound images corresponding to the anatomical region of interest, wherein each of the plurality of three-dimensional ultrasound images represents the anatomical region of interest from a different view angle. One or more candidate mass regions in each of the plurality of three-dimensional ultrasound images are identified. The method further includes determining one or more single-view features corresponding to each of the one or more candidate mass regions in each of the plurality of three-dimensional ultrasound images. For a candidate mass region of the one or more candidate mass regions in a three-dimensional ultrasound image of the plurality of three-dimensional ultrasound images, a similarity metric between the one or more single-view features corresponding to the candidate mass region and the one or more single-view features corresponding to the one or more candidate mass regions in the other three-dimensional ultrasound images of the plurality of three-dimensional ultrasound images is also determined. The candidate mass region is classified based at least on the similarity metric.

In accordance with an embodiment of the present specification, an imaging system is also presented. The imaging system includes an acquisition sub-system operatively coupled to a processing sub-system. The acquisition sub-system is configured to acquire a plurality of three-dimensional ultrasound images of the anatomical region of interest, wherein the plurality of three-dimensional ultrasound images is acquired at different view angles from the anatomical region of interest. The processing sub-system is configured to identify one or more candidate mass regions in each of the plurality of three-dimensional ultrasound images. The processing sub-system is also configured to determine one or more single-view features corresponding to each of the one or more candidate mass regions in each of the plurality of three-dimensional ultrasound images. The processing sub-system is further configured to determine, for a candidate mass region of the one or more candidate mass regions in a three-dimensional ultrasound image of the plurality of three-dimensional ultrasound images, a similarity metric between the one or more single-view features corresponding to the candidate mass region and the one or more single-view features corresponding to the one or more candidate mass regions in the other three-dimensional ultrasound images of the plurality of three-dimensional ultrasound images. The processing sub-system is also configured to classify the candidate mass region based at least on the similarity metric.

### DRAWINGS

These and other features, aspects, and advantages of the present specification will become better understood when the following detailed description is read with reference to the accompanying drawings in which like characters represent like parts throughout the drawings, wherein:

FIG. 1 is a diagrammatical illustration of an imaging system configured to detect lesions in an anatomical region of interest, in accordance with aspects of the present specification;

FIGS. 2(a), 2(b), and 2(c) are diagrammatical illustrations of different views of a breast;

FIG. 3 is a flow chart illustrating an exemplary method for detecting lesions in an anatomical region of interest, in accordance with aspects of the present specification;

FIG. 4 is a flow chart depicting an exemplary method for identifying candidate mass regions, in accordance with aspects of the present specification; and

FIGS. 5(a), 5(b), 5(c), and 5(d) are diagrammatical illustrations depicting an evolution of a candidate mass region at various steps of the method of FIG. 4.

### DETAILED DESCRIPTION

The specification may be best understood with reference to the detailed figures and description set forth herein. Various embodiments are described hereinafter with reference to the figures. However, those skilled in the art will readily appreciate that the detailed description given herein with respect to these figures is just for explanatory purposes as the method and the system extend beyond the described embodiments.

Conventionally, during the process of ultrasound scanning, the clinician, such as a radiologist or a sonographer tries to capture a view of a certain anatomy using a two-dimensional (2D) or three-dimensional (3D) ultrasound imaging system. The clinician may then examine the captured ultrasound images to manually detect the presence of lesion(s). On the other hand, 2D or 3D ultrasound systems with CAD based techniques aid in the automated detection of the lesions. However, the automated detection may result in an undesirable/unacceptable number of false positives. Also, the CAD based techniques entail separate analysis of each 3D ultrasound image.

The systems and methods for detecting lesions facilitate enhanced detection of the lesions. In particular, the lesions are detected based on information obtained from a combined analysis of a plurality of 3D ultrasound images. Moreover, use of the exemplary systems and methods aid in minimizing the number of false positives.

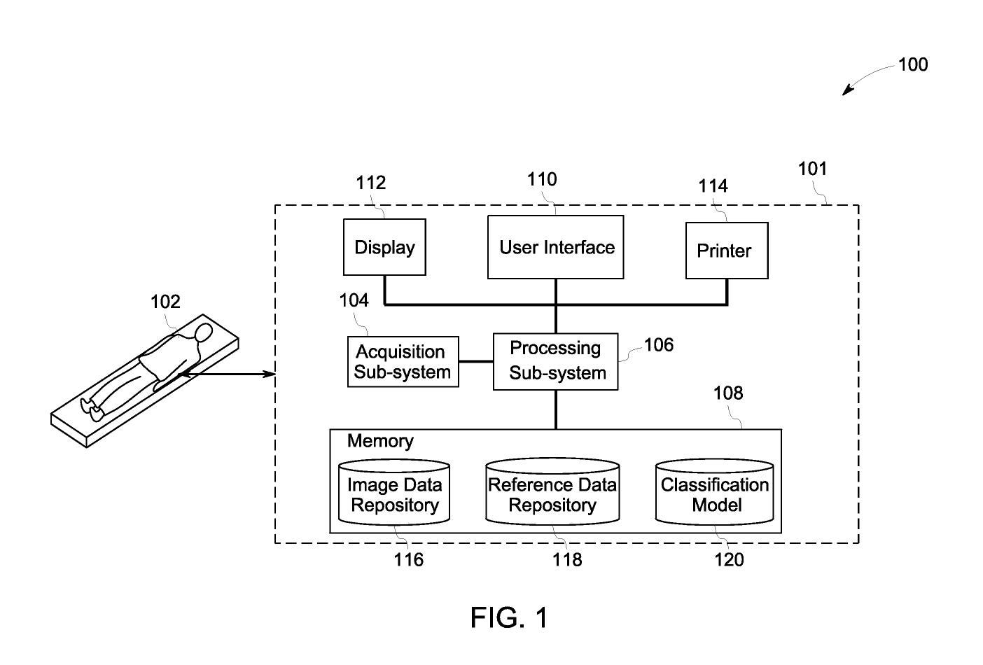

FIG. 1 is a diagrammatical illustration 100 of an imaging system 101 configured to detect lesions in an anatomical region of interest, in accordance with aspects of the present specification. Although the exemplary embodiments illustrated hereinafter describe the imaging system 101 in terms of an ultrasound imaging system, use of other types of imaging systems, such as, but not limited to, a computed tomography (CT) imaging system, a contrast enhanced ultrasound imaging system, an X-ray imaging system, an optical imaging system, a positron emission tomography (PET) imaging system, a magnetic resonance (MR) imaging system, and multi-modality imaging systems can also be contemplated without deviating from the scope of the specification. The multi-modality imaging systems may employ ultrasound imaging systems in conjunction with other imaging modalities, position-tracking systems or other sensor systems. For example, the multi-modality imaging system may include a PET imaging system-ultrasound imaging system.

In a presently contemplated configuration, the imaging system 101 may include an acquisition sub-system 104, a processing sub-system 106, memory 108, a user interface 110, and a display 112. In certain embodiments, the imaging system 101 may also include a printer 114. The memory 108 may include an image data repository 116, a reference data repository 118, and a classification model 120. The processing sub-system 106 may be operatively coupled to the acquisition sub-system 104, the memory 108, the user interface 110, the display 112, and/or the printer 114.

The acquisition sub-system 104 may be configured to acquire 3D ultrasound images of ananatomical region of interest of a patient 102. In certain embodiments, the acquisition of the image data may be customized based on one or more inputs provided by the clinician. The clinician may provide the inputs via use of the user interface 110. It may be noted that the anatomical region of interest may include any anatomy that can be imaged. For example, the anatomical region of interest may include breasts, a heart, an abdomen, a fetus, fetal features like a femur, a head, and the like, a chest, pelvis, hand(s), leg(s), and so forth. Although the present systems and methods are described in terms of detecting lesions in a breast, it may be noted that use of the present systems and methods for detecting lesions in other anatomical regions of interest is also envisaged, in accordance with the aspects of the present specification. Further, although the present specification is described with reference to the patient 102 being a human, it will be appreciated that the present systems and methods may also be applicable for detecting lesions in other living being without deviating from the scope of the present specification.

In one embodiment, the acquisition sub-system 104 may include a probe and/or a camera/sensor arrangement, also the acquisition sub-system 104 may be coupled to the patient 102. For example, the probe may include an invasive probe, a non-invasive probe, or an external probe, such as an external ultrasound probe, that is configured to aid in the acquisition of 3D ultrasound images. Also, the camera/sensor arrangement may be configured to acquire 3D ultrasound images of the breast at different view angles. To that end, the camera/sensor arrangement may include a 3D ultrasound camera/sensor mounted on a mechanical structure. The mechanical structure may be configured to adjust the position of the camera/sensor such that the camera/sensor is positioned at different view angles. In certain embodiments, the acquisition sub-system 104 may also include an actuator (e.g., a button) configured to trigger the acquisition of the 3D ultrasound images.

During the ultrasound examination, the acquisition sub-system 104 may be positioned at a suitable view angle with respect to the breast. The acquisition of the image data corresponding to a given view of the breast may be initiated. In one example, the acquisition of the image data corresponding to the breast may be automatically initiated. Alternatively, the acquisition of the image data may be manually initiated. A 3D ultrasound image, thus captured by the acquisition sub-system 104, may be stored in the image data repository 116. The step of capturing the 3D ultrasound images may be repeated at different view angles to acquire image data corresponding to the entire breast. In one embodiment, 3D ultrasound images may be captured such that each 3D ultrasound image has at least one portion that overlaps with one or more of the other 3D ultrasound images. These 3D ultrasound images may also be stored in the image data repository 116 for the further processing by the processing sub-system 106.

The processing sub-system 106 may be coupled to the acquisition sub-system 104 and configured to detect lesions in the anatomical region of interest based on analysis of the 3D ultrasound images. In certain embodiments, the processing sub-system 106 may be configured to retrieve the 3D ultrasound images of the breast from the image data repository 116. However, in other embodiments, the processing sub-system 106 may be configured to receive the 3D ultrasound images from the acquisition sub-system 104. In order to aid in the detection of the lesions, the processing sub-system 106 may be configured to identify one or more candidate mass regions in each of the plurality of 3D ultrasound images. The processing sub-system 106 may also be configured to determine one or more single-view features corresponding to each of the one or more candidate mass regions. In one example, the single-view features may include, but are not limited to, shape features, appearance features, texture features, posterior acoustic features, a distance to nipple, or combinations thereof. Some examples of the shape features may include, but are not limited to, a width, a height, a depth, a volume, a boundary, a height to width ratio, or combinations thereof. Also, some examples of the appearance features may include, but are not limited to, a mean intensity, a variance of the intensity, a contrast, a shade, energy, and entropy of a gray level co-occurrence matrix (GLCM), or combinations thereof.

Furthermore, the processing sub-system 106 may be configured to analyze each candidate mass region of the one or more candidate mass regions in a 3D ultrasound image of the plurality of 3D ultrasound images. In particular, each candidate mass region may be analyzed to determine a similarity metric between the one or more single-view features corresponding to the candidate mass region and the one or more single-view features corresponding to the one or more candidate mass regions in other 3D ultrasound images. The similarity metric may be indicative of the similarity between the one or more single-view features corresponding to the candidate mass region and the one or more single-view features corresponding to the one or more candidate mass regions in other 3D ultrasound images.

The processing sub-system 106 may also be configured to classify the candidate mass region based at least on the determined similarity metric. By the way of example, the candidate mass region may be classified as a lesion based on the determined similarity metric. In accordance with the aspects of the present specification, the processing sub-system 106 may be configured to classify the candidate mass region based on reference data and the classification model 120. In certain embodiments, the reference data may be stored in the reference data repository 118. The reference data may include information such as various manually classified reference 3D ultrasound images, and threshold values of similarity metric for the one or more single-view features corresponding to various candidate mass regions in the sample 3D ultrasound images. For example, if a threshold value for a similarity metric of a single view feature such as a distance to nipple has a value of 98% then for all candidate mass regions having a similarity metric value (of the distance to nipple feature) equal to or greater than 98% may be classified as a lesion. In one embodiment, the threshold values may either be manually or automatically set based on the manual classification of the reference images.

In one embodiment, the classification model 120 may be developed based on the reference data. According to the embodiments of the present specification, the classification model 120 may be implemented as a Random Forest (RF) classifier, a Support Vector Machine (SVM) classifier, or a combination thereof. It may be noted that, the present technique of detecting lesions may also be based on other learning techniques and types of the classification model 120.

The processing sub-system 106 may be implemented as hardware elements such as circuit boards with digital signal processors or as software running on a processor such as a commercial, off-the-shelf personal computer (PC), or a microcontroller. The processing sub-system 106 may also be realized as single processor or multi-processor system capable of executing the method of detecting lesions. The single processor system may be based on multi-core or single-core architecture.

The user interface 110 of the imaging system 101 may include a human interface device (not shown) configured to aid the clinician in acquiring the 3D ultrasound images through the acquisition sub-system 104. Furthermore, in accordance with the aspects of the present specification, the user interface 110 may be configured to aid the clinician in navigating through the 3D ultrasound images. Additionally, the user interface 110 may also be configured to aid in performing various other functions, such as, but not limited to, manipulating, annotating, organizing the displayed 3D ultrasound images, and issuing a print command. The human interface device may include a mouse-type device, a trackball, a joystick, a stylus, a voice recognition system, or a touch screen configured to facilitate the capturing and manipulating by the clinician.

Also, the display 112 may be configured to display a current ultrasound view of the breast being imaged, thereby aiding the clinician in capturing an image of the breast at various view angles. In accordance with aspects of the present specification, the display 112 may also be configured to display the 3D ultrasound images captured by acquisition sub-system 104.

In certain embodiments, the functionalities of the user interface 110 and the display 112 may also be combined. For example, a touch screen can be configured to function as both the user interface 110 and the display 112. Moreover, the printer 114 may be used to print an image with or without any annotation.

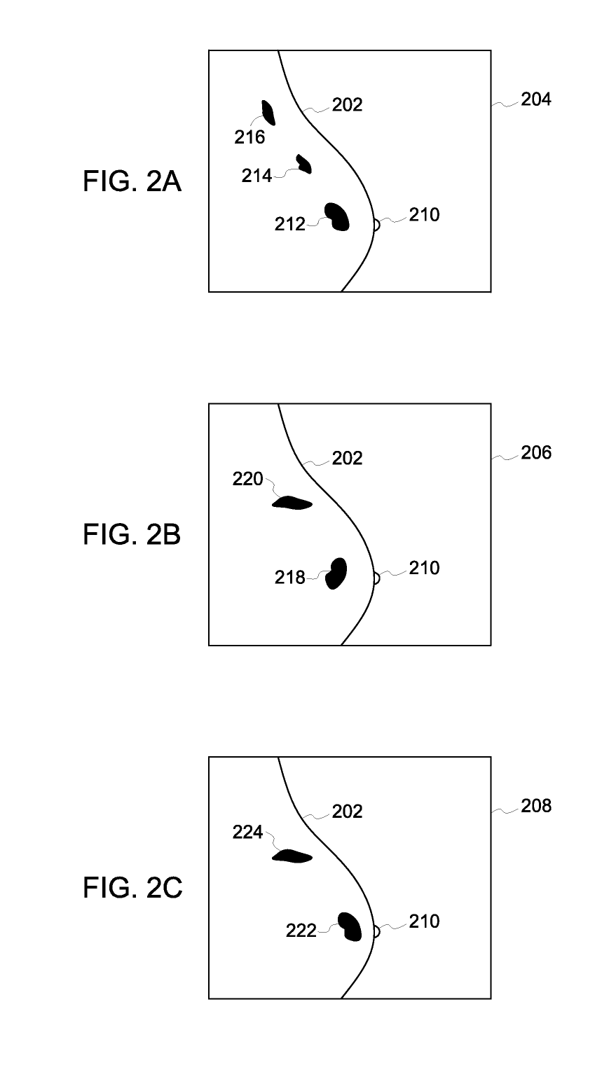

FIGS. 2(a), 2(b), and 2(c) are diagrammatical illustrations of different views of a breast 202. FIG. 2(a) is a diagrammatical representation of a first view 204 of the breast 202. Similarly, FIG. 2(b) is a diagrammatical representation of a second view 206 of the breast 202, while FIG. 2(c) is a diagrammatical representation of a third view 208 of the breast 202. The first view 204, the second view 206, and the third view 208 may represent 3D ultrasound images of the breast 202 that are acquired at different view angles. Collectively, the first view 204, the second view 206, and the third view 208 may be hereinafter interchangeably referred to as a plurality of 3D ultrasound images, 3D ultrasound images, images, or image data.

In one embodiment, the 3D ultrasound images 204, 206, 208 may be respectively representative of a medio-lateral oblique (MLO) view, a cranio-caudal (CC) view, and a rolled CC view of the breast 202. The above views are exemplary and the 3D ultrasound images acquired from various other view angles including, but not limited to, a lateromedial (LO) view, a mediolateral (ML) view, a spot compression view, a cleavage view, a true lateral view, a lateromedial view, a lateromedial oblique view, a late mediolateral view, a step oblique view, a magnification view, an exaggerated craniocaudal view, an axillary view, a tangential view, a reversed CC view, and a bull's-eye CC view may also be used without deviating from the scope of the present specification. Although, the above mentioned views are generally applicable for imaging breasts, in one embodiment, for imaging other anatomical regions of interest, 3D ultrasound images may also be acquired by positioning the acquisition sub-system 104 at different angular positions with respect to the anatomical region of interest.

In the plurality of 3D ultrasound images 204, 206, and 208, regions marked by reference numerals 212-224 are generally representative of candidate mass regions. In particular, reference numerals 212, 214, and 216 are representative of candidate mass regions in FIG. 2(a). Similarly, reference 218 and 220 are representative of candidate mass regions in FIG. 2(b), while the candidate mass regions in FIG. 2(c) are generally represented by reference numerals 222 and 224. One or more of the candidate mass regions 212-224 may be representatives of lesions. Also, reference numeral 210 may be representative of a nipple. Moreover, the candidate mass regions 212, 218, and 222 that respectively correspond to images 204, 206, and 208, appear to be substantially similar with respect to their shapes, sizes and relative positions with respect to the nipple 210. Accordingly, it may be assumed that the candidate mass regions 212, 218, and 222 represent a first breast mass in different views. Similarly, the candidate mass regions 214, 220, and 224 that respectively correspond to images 204, 206, and 208, appear to be substantially similar with respect to their shapes, sizes and relative positions with respect to the nipple 210. Accordingly, it may be assumed that the candidate mass regions 214, 220, and 224 represent a second breast mass (i.e., different than the first breast mass) in different views. The candidate mass region 216 in the image 204 does not have a matching candidate mass region in the other 3D ultrasound images 206 and 208.

In accordance with the aspects of the present specification, as a lesion appears similar in the size, shape and position in the plurality of 3D ultrasound images 204, 206, and 208, the candidate mass regions 212, 218, and 222, and 214, 220, and 224 may be considered as lesions. Also, it may be assumed that the candidate mass region 216 is representative of an artifact or a temporary volume observed due to external pressure applied on the breast 202. Accordingly, the imaging system 101 (see FIG. 1) for the automated detection of lesions may be configured to detect lesions in the breast 202 based on a similarity metric between the single view features among the plurality of 3D ultrasound images 204, 206, and 208.

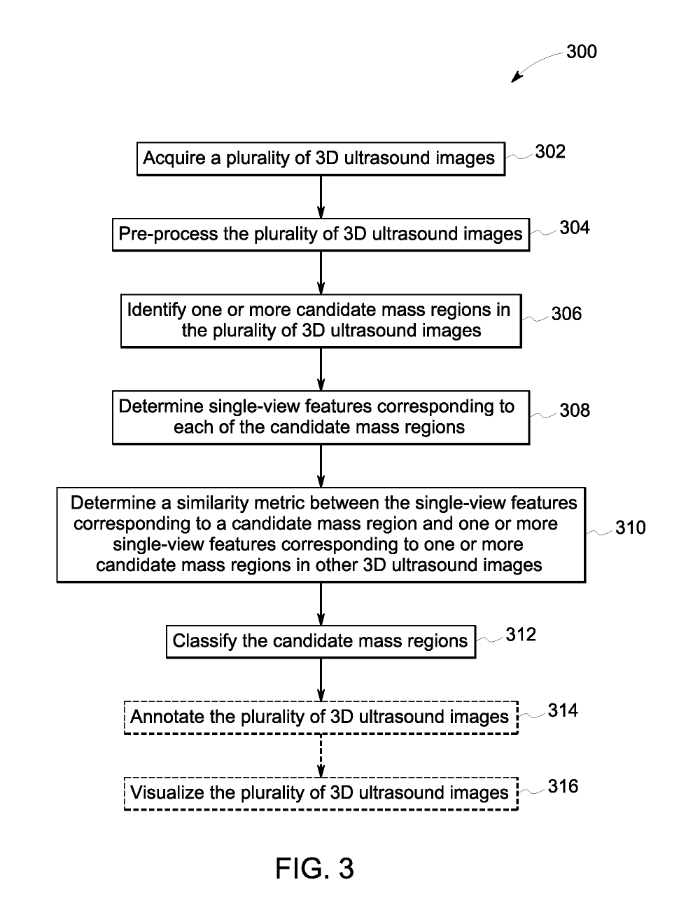

FIG. 3 is a flow chart 300 illustrating an exemplary method for detecting lesions in an anatomical region of interest, in accordance with aspects of the present specification. The method of FIG. 3 may be described with respect to the elements of FIGS. 1 and 2.

At step 302, a plurality of 3D ultrasound images, such as the 3D ultrasound images 204, 206, and 208 of an anatomical region of interest, such as the breast 202 may be acquired. In one embodiment, the acquisition sub-system 104 may be used to aid in the acquisition of the 3D image data. As previously noted, in order to acquire the plurality of 3D ultrasound images 204, 206, and 208, the acquisition sub-system 104 may be positioned at a suitable view angle (e.g., at a position suitable to capture an MLO view) with respect to the breast 202 to capture the MLO view of the breast 202. A 3D ultrasound image such as the 3D ultrasound image 204 thus captured may be stored in the image data repository 116. This procedure to capture a plurality of 3D ultrasound images such as 3D ultrasound images 206 and 208 may be repeated by positioning the acquisition sub-system 104 at different view angles (e.g., at a position suitable to capture a CC view and a rolled CC view) such that image data corresponding to the entire breast 202 may be acquired. In one embodiment, each of the plurality of 3D ultrasound images 204, 206, 208 is acquired such that each image 204, 206, 208 includes at least a portion that overlaps with one or more of the other 3D ultrasound images. The plurality of 3D ultrasound images 204, 206, 208 is stored in the image data repository 116 for further processing by the processing sub-system 106.

Furthermore, at step 304, the plurality of 3D ultrasound images 204, 206, 208 may be pre-processed by processing sub-system 106. In one embodiment, for example, the plurality of 3D ultrasound images 204, 206, 208 may be processed to minimize noise such as speckle. Such pre-processing aids in improving the clarity of the plurality of 3D ultrasound images 204, 206, and 208. By the way of an example, the processing sub-system 106 may be configured to employ speckle minimization techniques such as, but not limited to, statistical segmentation of images, Bayesian multi-scale methods, filtering techniques, maximum likelihood technique, and the like to minimize the speckle noise in the 3D ultrasound images 204, 206 and 208.

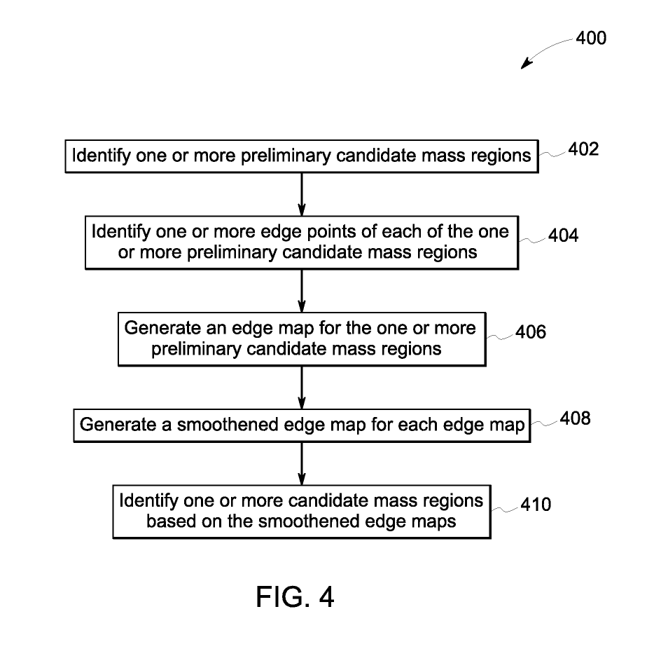

At step 306, one or more candidate mass regions such as the candidate mass regions 212-224 may be identified in each of the plurality of 3D ultrasound images 204, 206, and 208. The candidate mass regions 212-224 may be representative of masses/volumes that may be probable lesions. The method of identifying the candidate mass regions will be described in greater detail with reference to FIG. 4.

Once the candidate mass regions 212-224 are identified, single-view features corresponding to each of the candidate mass regions 212-224 may be determined, as indicated by step 308. For example, the single-view features may include features such as shape features, appearance features, texture features, posterior acoustic feature, distance to nipple, and the like.

In one embodiment, the processing sub-system 106 may be configured to determine the single view features corresponding to each candidate mass region 212-224. The processing sub-system 106 may be configured to determine the shape features such as the width, the height, the depth, and the volume of each of the candidate mass regions 212-224. The processing sub-system 106 may also be configured to determine appearance features such as contrast, shade, energy, entropy of the GLCM, the mean and the variance of the intensity in each of the candidate mass regions 212-224. Further, the processing sub-system 106 may also be configured to determine a texture of each of the candidate mass regions 212-224. In one embodiment, the texture may be determined based on a Sobel operator. Moreover, in one embodiment, the Sobel operator may be applied to each of the candidate mass regions 212-224 in an anterior-posterior direction and an inferior-superior direction. For each of the plurality of 3D ultrasound images 204, 206, and 208, the mean and the variance of the intensity within the candidate mass regions 212-224 may be computed. These features may be representatives of the Sobel operator features. Furthermore, various other single view features such as a posterior acoustic feature, a mass boundary, a normalized radial gradient (NRG), and a minimum side difference (MSD) may also be computed.

At step 310, for a candidate mass region, the processing sub-system 106 may be configured to determine a similarity metric between the single-view features corresponding to the candidate mass region and one or more single-view features corresponding to one or more candidate mass regions in other 3D ultrasound images of the plurality of 3D ultrasound images. For example, the processing sub-system 106 may be configured to determine a similarity metric between the single-view features corresponding to the candidate mass region 212 and the single-view features corresponding to the other candidate mass regions in other 3D ultrasound images (e.g., the candidate mass regions 218 and 220 in the 3D ultrasound image 206; and the candidate mass regions 222 and 224 in the 3D ultrasound image 208). In one embodiment, step 310 may be repeated for the remaining candidate mass regions.

It may be noted that, if a single breast is scanned at N different views (i.e., N number of 3D ultrasound images have been acquired) with Mᵢ candidate mass regions identified in each view, candidate mass regions in view i may be represented as, L_{i,1}, L_{i,2}, L_{i,3}, . . . , L_{i,M}_{ᵢ}.

A single-view feature x(i,j) extracted from L_{i,j}, where jε(1, 2, . . . , Mᵢ), may be compared with a single-view feature x(k,l) extracted from L_{k,l} in other views, where k≠i,kε(1, 2, . . . , N) and lε(1, 2, . . . , Mₖ). An absolute difference Δx(i,j,k,l) may be determined based on the comparison:

Δx(i,j,k,l)=|x(i,j)−x(k,l)| (1)

Once the absolute differences corresponding to all the single-view features are determined, a minimum value (xₘᵥ(i,j)) of the absolute difference may be determined using:

$${x}_{mv} ( i , j ) = {min}_{{k \neq i ,}_{l ∈ ( 1 , 2 , … , {M}_{k} )}} \vert x ( i , j ) - x ( k , l ) \vert \qquad ( 2 )$$

It may be noted that in comparison to the candidate mass regions caused by artifacts (e.g., the candidate mass region 216), the candidate mass regions that represent lesions (hereinafter alternatively referred to as actual masses) have a higher probability of appearing in more than one view. Therefore, the minimum value of the absolute difference (xₘᵥ(i,j)) for each feature is smaller for an actual mass, such as, a mass represented by the candidate mass regions 212, 218, and 222; and a mass represented by the candidate mass regions 214, 220, and 224.

In one embodiment, the comparison of step 310 may also be performed corresponding to a subset of the single-view features. In one embodiment, the entropy of GLCM, the posterior acoustic feature, the lesion boundary, the Sobel operator features, and the distance from a candidate mass region to the nipple may be considered for the analysis at step 310. However, a single-view feature, such as the mean intensity, that tends to share similar characteristics between actual masses and masses caused by artifacts in different views, may not be used for determining the similarity metric.

Moreover, at step 312, the candidate mass regions may be classified at least based on the similarity metric determined at step 310. For example, if the value of the absolute difference Δx(i,j,k,l) corresponding to one or more single-view features associated with candidate mass regions L_{i,1} and L_{i,2} (e.g., the candidate mass regions 212 and 214 in the 3D ultrasound image 204) has a minimum value, then L_{i,1} and L_{i,2} may be classified as lesions. However, since the candidate mass region 216 appears only in the 3D ultrasound image 204, the value of the absolute difference Δx(i,j,k,l) associated with the candidate mass region 216 may not have a minimum value. Thus, the candidate mass region 216 may not be classified as lesion.

In one embodiment, the processing sub-system 106 may be employed to classify the candidate mass regions 212-224. In particular, in accordance with the aspects of the present specification, the processing sub-system 106 may be configured to classify the candidate mass regions 212-224 based on the classification model 120. More particularly, the classification model 120 may be used to determine whether a candidate mass region may be classified as a lesion or not based on the values of similarity metric (e.g., the values of Δx(i,j,k,l) and xₘᵥ(i,j)) determined at step 310. In another embodiment, the single-view features may also be used to aid in the classification.

At step 314, the plurality of 3D ultrasound images 204, 206, 208 may be annotated to indicate the candidate mass regions that have been classified as lesions at step 312. In one embodiment, the processing sub-system 106 may be employed to annotate the candidate mass regions in the plurality of 3D ultrasound images 204, 206, 208. The candidate mass regions that have been identified as lesions may be annotated accordingly. For example, in the 3D ultrasound image 204, the candidate mass regions 212 and 214 may be marked as lesions. The candidate mass regions 212 and 214 may be annotated with an indicator such as, but not limited to, a rectangle, a square, a circle, an ellipse, an arrow, or any other shape, without deviating from the scope of the present specification. In another embodiment, the annotation may include embedded text that indicates a location/presence of lesions in the image. In yet another embodiment, the annotation may include use of shaped indicators and embedded text. Furthermore, if no lesion is detected, a text indicating absence of lesions may be embedded in the plurality of 3D ultrasound images 204, 206, and 208. In one embodiment, step 314 may be optional.

In addition, at step 316, the plurality of annotated 3D ultrasound images 204, 206, 208 may be visualized on a display such as the display 112. In one embodiment, one or more of the plurality of 3D ultrasound images 204, 206, 208 may be printed. In one embodiment, step 316 may be optional.

FIG. 4 is a flow chart 400 depicting an exemplary method for identifying candidate mass regions, in accordance with aspects of the present specification. In particular, the flow chart 400 illustrates details of step 306 of the flow chart 300 of FIG. 3.

At step 402, one or more preliminary candidate mass regions in a plurality of 3D ultrasound images may be identified. A preliminary candidate mass region may be representative of a volume that may be a probable candidate mass region. In one embodiment, a voxel based technique may be used to identify the one or more preliminary candidate mass regions. It may be noted that the preliminary candidate mass region may not have a clearly defined boundary.

Furthermore, at step 404, one or more edge points of each of the one or more preliminary candidate mass regions may be identified. In one embodiment, for example, the processing sub-system 106 may be configured to perform a directional search from a determined location in the preliminary candidate mass region to identify the one or more edge points. In one embodiment, the determined location may be the center of the preliminary candidate mass region. By way of an example, to perform the directional search for the edge points, a set of rays in each direction may be created from the center of the preliminary candidate mass region. One or more points on each ray may be inspected. In one embodiment, for regions within the preliminary candidate mass region, all the points on the ray may be considered. In another embodiment, for the regions that are outside the preliminary candidate mass region, only points within a determined distance from an approximate boundary of the preliminary candidate mass region in the direction of ray may be considered. Furthermore, in one embodiment, in considering the points with an increasing gradient, a point having a maximum gradient magnitude may be selected as the edge point in this direction. More particularly, the increasing gradient constraint may be enforced because the regions within the preliminary candidate mass region tend to have lower intensities than the regions that are outside of the candidate mass regions.

Subsequently, at step 406, an edge map may be generated for each of the one or more preliminary candidate mass regions. The edge points corresponding to a preliminary candidate mass region may be indicative of an edge of the preliminary candidate mass region. In one embodiment, in order to determine the edge map, the processing sub-system 106 may be configured to apply Gaussian blur on the edge points so that dense edge points (e.g., edge points that are located in close proximity of one another) produce higher intensities and sparse edge points (e.g., edge points that are located far from one another) produce lower intensities on the edge map.

Moreover, at step 408, a smoothened edge map corresponding to each edge map may be generated. The search for the edge points is performed from the determined location in the preliminary candidate mass region (e.g., from the center of the preliminary candidate mass region). Also, edge points get sparser with larger radii. Therefore, a compensation/normalization of the distance of the edge point to the origin of the rays (e.g., the center of the preliminary candidate mass region) is made in order to smoothen the edge map. In one embodiment, from each edge point on the edge map, the distance to the origin of the ray is calculated. In one example, the compensation may entail multiplying the square of this distance with an intensity value of a corresponding edge point.

At step 410, one or more candidate mass regions may be identified based on the smoothened edge maps generated at step 408. The one or more candidate mass regions may be identified by determining a boundary of each of the one or more preliminary candidate mass regions. In one embodiment, the boundary may be determined based on the smoothened edge map. The preliminary candidate mass region with the clearly defined boundary may be referred to as the candidate mass region. The processing sub-system 106 may be configured to employ a 3D Geodesic Active Contours (GAC) technique to determine the candidate mass region using the smoothened edge map. In particular, a level set function (u) may be used to represent the candidate mass region. Furthermore, in one embodiment, using the level set function (u) and the GAC technique, the boundary of the candidate mass region may be evolved based on the image intensity of the preliminary candidate mass region. The boundary of the candidate mass region may be represented as:

$$\frac{\partial u}{\partial t} = g ( I ) \vert ∇ u \vert k + ∇ g ( I ) \cdot Δ u \qquad ( 3 )$$

where, g(I) is a positive decreasing edge detector (PDED) function, and I is the image intensity.

In one embodiment, the PDED function g(I) may be represented as:

$$g ( I ) = \frac{1}{1 + {e}^{[ \frac{( {E}_{m} - β )}{α} ]}} \qquad ( 4 )$$

where, Eₘ represents the smoothened edge map, and (∇*G) represents a derivative of a Gaussian operator G, and α and β are constants.

In accordance with the aspects of the present specification, the PDED function g(I) may be determined based on the smoothened edge map Eₘ as opposed to using a derivative of the Gaussian operator (∇*G) as the inhomogeneity and/or loosely defined boundary of the preliminary candidate mass region, (∇*G)(I) may impede the determination of sharp edges. Thus, directly evolving the candidate mass regions based on the preliminary candidate mass regions (which are obtained after applying the voxel based technique) may fail as the segmentation may easily be trapped in a local maxima. Therefore, the use of the smoothened edge map Eₘ in determining the boundary of the candidate mass region aids in the detection of sharp and clear boundaries.

Moreover, as the smoothened edge map Eₘ is used while applying GAC, some details in the ultrasound image may be lost. Accordingly, step 410 may be repeated using the boundary of equation 3 as initialization.

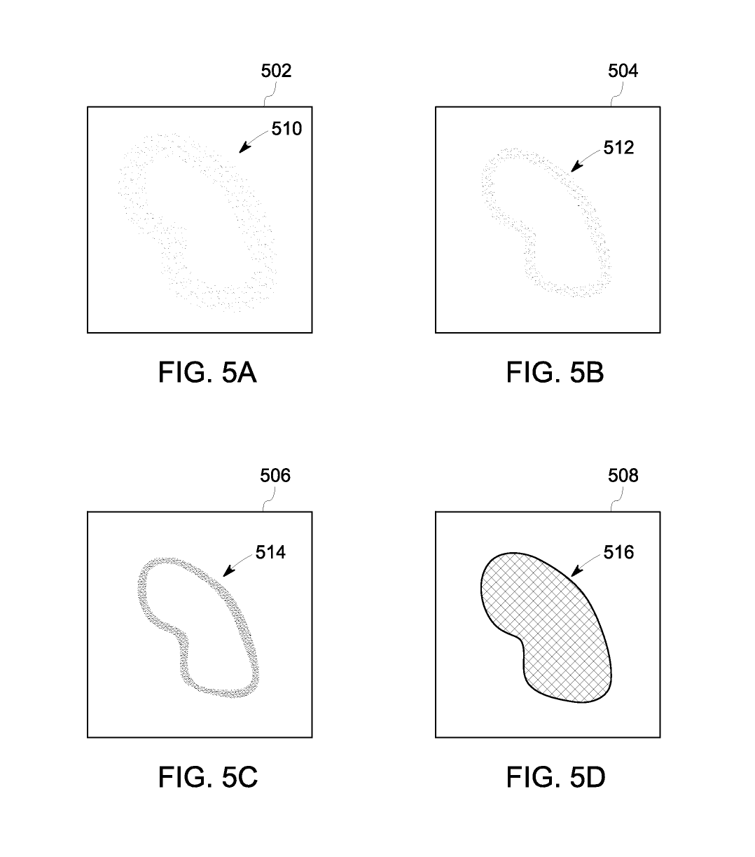

FIGS. 5(a)-5(d) represent diagrammatical illustrations 502, 504, 506, 508 that depict an evolution of the candidate mass region 212 of FIG. 2(a) at various steps of the method of FIG. 4.

In the diagrammatical illustration 502, reference numeral 510 may represent a preliminary candidate mass region. The preliminary candidate mass region 510 may have a loosely defined boundary formed by multiple points. It may be noted that the boundary of the preliminary candidate mass region 510 is not clearly evident as the points are sparse. In one embodiment, the preliminary candidate mass region 510 may be obtained at step 402.

Furthermore, in the diagrammatical illustration 504, reference numeral 512 may represent a preliminary candidate mass region with identified edge points. In one embodiment, the edge points may be obtained at step 404.

Also, in the diagrammatical illustration 506, reference numeral 514 may represent the preliminary candidate mass region with a smoothened edge map. In one embodiment, the smoothened edge map may be generated at step 408. Due to the smoothened edge map, the boundary of the preliminary candidate mass region 514 may appear sharper than the boundary of the preliminary candidate mass region 510 obtained at step 402.

Moreover, in the diagrammatical illustration 508, reference numeral 516 may represent the candidate mass region which is determined from the preliminary candidate mass region 514. In one embodiment, the candidate mass region 516 may be identified after processing the preliminary candidate mass region 514 with the smoothened edge map generated at step 410. In one embodiment, the candidate mass region 516 may represent the candidate mass region 212 of FIG. 2(a).

The system, modules, and sub-modules have been illustrated and explained to serve as examples and should not be considered limiting in any manner. The variants of the above disclosed system elements, modules and other features and functions, or alternatives thereof, may be combined to create many other different systems or applications.

The method and system for the automated detection of lesions described hereinabove greatly reduce the number of false positive detections as the system and method not only consider the single-view features but also take into account the interdependency/similarity between the single-view features in multiple 3D ultrasound images. Further, as compared to 2D images obtained by mammography or ultrasound examination, the 3D images have additional depth information. Therefore, the single-view features derived from the 3D images can better describe the lesion. Moreover, according to the aspects of the present specification, the single-view features derived from a single 3D image are compared with the single-view features derived from other 3D images during multi-view analysis. Therefore, the accuracy of detection of the lesions is consequently enhanced while the false positive detections are minimized.

Furthermore, in order to determine sharp boundaries of the candidate mass regions, the exemplary method described herein above utilizes the smoothened edge map as opposed to the use of the derivative of the intensity image in the currently available techniques (e.g., the GAC technique). The smoothened edge map, which is derived from the edge points identified by the directional search, aids in the detection of sharp boundaries.

Any of the foregoing steps and/or system modules may be suitably replaced, reordered, or removed, and additional steps and/or system modules may be inserted, depending on the needs of a particular application, and that the systems of the foregoing embodiments may be implemented using a wide variety of suitable processes and system modules and are not limited to any particular computer hardware, software, middleware, firmware, microcode, etc.

Furthermore, the foregoing examples, demonstrations, and process steps such as those that may be performed by the imaging system may be implemented by suitable code on a processor-based system, such as a general-purpose or special-purpose computer. Different implementations of the systems and methods may perform some or all of the steps described herein in different orders, parallel, or substantially concurrently. Furthermore, the functions may be implemented in a variety of programming languages, including but not limited to C++ or Java. Such code may be stored or adapted for storage on one or more tangible, computer readable media, such as on data repository chips, local or remote hard disks, optical disks (that is, CDs or DVDs), memory or other media, which may be accessed by a processor-based system to execute the stored code. Note that the tangible media may comprise paper or another suitable medium upon which the instructions are printed. For instance, the instructions may be electronically captured via optical scanning of the paper or other medium, then compiled, interpreted or otherwise processed in a suitable manner if necessary, and then stored in the data repository or memory.

It will be appreciated that variants of the above disclosed and other features and functions, or alternatives thereof, may be combined to create many other different systems or applications. Various unanticipated alternatives, modifications, variations, or improvements therein may be subsequently made by those skilled in the art and are also intended to be encompassed by the following claims.

1. A method for detecting a lesion in an anatomical region of interest, the method comprising:
receiving a plurality of three-dimensional ultrasound images corresponding to the anatomical region of interest, wherein each of the plurality of three-dimensional ultrasound images represents the anatomical region of interest from a different view angle;
identifying one or more candidate mass regions in each of the plurality of three-dimensional ultrasound images;
determining one or more single-view features corresponding to each of the one or more candidate mass regions in each of the plurality of three-dimensional ultrasound images;
determining, for a candidate mass region of the one or more candidate mass regions in a three-dimensional ultrasound image of the plurality of three-dimensional ultrasound images, a similarity metric between the one or more single-view features corresponding to the candidate mass region and the one or more single-view features corresponding to the one or more candidate mass regions in the other three-dimensional ultrasound images of the plurality of three-dimensional ultrasound images; and
classifying the candidate mass region based at least on the similarity metric.

2. The method of claim 1, wherein the anatomical region of interest is a breast.

3. The method of claim 2, further comprising acquiring the plurality of three-dimensional ultrasound images of the breast at different view angles.

4. The method of claim 3, wherein the different view angles comprise a cranio-caudal (CC) view, a mediolateral-oblique (MLO) view, a lateromedial (LO) view, a mediolateral (ML) view, a spot compression view, a cleavage view, or combinations thereof.

5. The method of claim 3, wherein acquiring the plurality of three-dimensional ultrasound images comprises acquiring each of the plurality of three-dimensional ultrasound images such that a three-dimensional ultrasound image overlaps with one or more of other three-dimensional ultrasound images.

6. The method of claim 1, further comprising pre-processing the plurality of three-dimensional ultrasound images to minimize noise.

7. The method of claim 1, further comprising determining one or more preliminary candidate mass regions in each of the plurality of three-dimensional ultrasound images using a voxel based technique.

8. The method of claim 7, wherein identifying the one or more candidate mass regions comprises:
identifying one or more edge points of each of the one or more preliminary candidate mass regions by directionally searching for the one or more edge points from a determined location in each of the one more preliminary candidate mass regions;
generating an edge map for each of the one or more preliminary candidate mass regions based on the corresponding one or more edge points; and
determining a boundary of each of the one more preliminary candidate mass regions to identify the one or more candidate mass regions, wherein the boundary is determined based on a corresponding edge map.

9. The method of claim 8, further comprising generating a smoothened edge map for each of preliminary candidate mass regions by compensating for distances of the one or more edge points on the edge map to the determined location in each the one or more preliminary candidate mass regions.

10. The method of claim 9, wherein determining the boundary of each of the one more preliminary candidate mass regions comprises processing the smoothened edge map via a geodesic active contour.

11. The method of claim 9, wherein compensating for the distances comprises processing the smoothened edge map by a Gaussian blur.

12. The method of claim 1, wherein the one or more single-view features comprise a shape feature, an appearance feature, a texture feature, a posterior acoustic feature, a distance to nipple, or combinations thereof.

13. The method of claim 12, wherein the shape feature comprises a width, a height, a depth, a volume, a boundary, a height to width ratio of each of the one or more candidate mass regions in each of the plurality of three-dimensional ultrasound images, or combinations thereof.

14. The method of claim 12, wherein the appearance feature comprises a mean intensity, a variance of the intensity, a contrast, a shade, an energy, an entropy of a gray level co-occurrence matrix (GLCM) of each of the one or more candidate mass regions in each of the plurality of three-dimensional ultrasound images, or combinations thereof.

15. The method of claim 1, wherein classifying the candidate mass region comprises using a Random Forest classifier, a Support Vector Machine classifier, or a combination thereof.

16. A system for imaging an anatomical region of interest, the system comprising:
an acquisition sub-system configured to acquire a plurality of three-dimensional ultrasound images of the anatomical region of interest, wherein the plurality of three-dimensional ultrasound images is acquired at different view angles from the anatomical region of interest;
a processing sub-system operatively coupled to the acquisition sub-system and configured to:
identify one or more candidate mass regions in each of the plurality of three-dimensional ultrasound images;
determine one or more single-view features corresponding to each of the one or more candidate mass regions in each of the plurality of three-dimensional ultrasound images;
determine, for a candidate mass region of the one or more candidate mass regions in a three-dimensional ultrasound image of the plurality of three-dimensional ultrasound images, a similarity metric between the one or more single-view features corresponding to the candidate mass region and the one or more single-view features corresponding to the one or more candidate mass regions in the other three-dimensional ultrasound images of the plurality of three-dimensional ultrasound images; and
classify the candidate mass region based at least on the similarity metric.

17. The system of claim 16, wherein the processing sub-system is configured to minimize speckle noise in the plurality of three-dimensional ultrasound images.

18. The system of claim 16, wherein the processing sub-system is configured to determine one or more preliminary candidate mass regions in each of the plurality of three-dimensional ultrasound images using a voxel based technique.

19. The system of claim 18, wherein the processing sub-system is further configured to:
identify one or more edge points of each of the one or more preliminary candidate mass regions by directionally searching for the one or more edge points from the center of each of the one more preliminary candidate mass regions;
generate an edge map for each of the one or more preliminary candidate mass regions based on the corresponding one or more edge points; and
determine a boundary of each of the one more preliminary candidate mass regions to identify the one or more candidate mass regions, wherein the boundary is determined based on the edge map.

20. The system of claim 16, wherein the processing sub-system is further configured to classify the candidate mass region using a Random Forest classifier, a Support Vector Machine classifier, or a combination thereof.

21. A non-transitory computer readable media storing an executable code to perform method of:
receiving a plurality of three-dimensional ultrasound images corresponding to the breast, wherein each of the plurality of three-dimensional ultrasound images represents the breast from a different view angle;
identifying one or more candidate mass regions in each of the plurality of three-dimensional ultrasound images;
determining one or more single-view features corresponding to each of the one or more candidate mass regions in each of the plurality of three-dimensional ultrasound images;
determining, for a candidate mass region of the one or more candidate mass regions in a three-dimensional ultrasound image of the plurality of three-dimensional ultrasound images, a similarity metric between the one or more single-view features of the candidate mass region and the one or more single-view features corresponding to the one or more candidate mass regions in the other three-dimensional ultrasound images of the plurality of three-dimensional ultrasound images; and
classifying the candidate mass region based at least on the similarity metric.
